(19) United States
(12) Patent Application Publication
(10) Pub. No.: US 2024/0156585 A1
(43) Pub. Date: May 16, 2024

(54) ENDOVASCULAR DEVICES

(71) Applicant: Merit Medical Systems, Inc., South Jordan, UT (US)

(72) Inventors: Mattias Nielsen, North Salt Lake, UT (US); John Hall, Bountiful, UT (US); Christopher Cindrich, Highland, UT (US)

(21) Appl. No.: 18/508,005

(22) Filed: Nov. 13, 2023

Related U.S. Application Data
(60) Provisional application No. 63/383,877, filed on Nov. 15, 2022.

(51) Int. Cl. A61F 2/06

(52) U.S. Cl. CPC A61F 2/06; A61F 2002/072

(57) ABSTRACT

A stent for treating a patient that may comprise a body with a first end portion with a first opening and a second end portion with a second opening opposite the first opening. A stent may include a lumen that extends from the first opening to the second opening. The stent includes a single fenestration disposed in a sidewall of the body between the first opening and the second opening. The single fenestration is disposed in a center portion of the body of the stent in a length direction.

### RELATED APPLICATIONS

This application claims priority to U.S. Provisional Application No. 63/383,877, filed on Nov. 15, 2022 and titled, “Endovascular Devices,” which is hereby incorporated by reference in its entirety.

### TECHNICAL FIELD

This invention relates generally to medical devices. More specifically, some embodiments relate to methods and apparatus for medical appliances or endovascular prosthesis, such as stents and stent-grafts.

### BRIEF DESCRIPTION OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1 illustrates a stenosis or blockage in a common iliac artery of a patient.

FIG. 2A is a perspective view of a stent according to one embodiment of the present disclosure.

FIG. 2B is a perspective view of a stent according to one embodiment of the present disclosure.

FIG. 3A is a perspective view of a trunk stent according to one embodiment of the present disclosure.

FIG. 3B is a perspective view of a trunk stent according to one embodiment of the present disclosure.

FIG. 3C is a perspective view of a trunk stent according to one embodiment of the present disclosure.

FIG. 4 is a perspective view of a stent system with a stent and a trunk stent coupled to the stent according to one embodiment of the present disclosure.

FIG. 5 illustrates a process of implanting a stent system in a common iliac artery of a patient according to one embodiment of the present disclosure.

FIG. 6 illustrates a stent implanted in the common iliac artery of the patient of FIG. 5

FIG. 7 illustrates a process of implanting a trunk stent in a single fenestration of the stent of FIG. 6.

FIG. 8 illustrates the trunk stent coupled to the stent of FIG. 7 in the common iliac artery of the patient.

FIG. 9 illustrates two stent systems implanted in a common iliac artery of a patient according to one embodiment of the present disclosure.

FIG. 10 illustrates a stent implanted in a common iliac artery of a patient according to one embodiment of the present disclosure.

FIG. 11 illustrates a stent implanted in a common iliac artery of a patient according to one embodiment of the present disclosure.

FIG. 12A illustrates a stent in a linear configuration according to one embodiment of the present disclosure.

FIG. 12B illustrates the stent of FIG. 12A in a curved configuration.

FIG. 13A illustrates a stent in a linear configuration according to one embodiment of the present disclosure.

FIG. 13B illustrates the stent of FIG. 13A in a curved configuration.

### DETAILED DESCRIPTION OF THE INVENTION

Medical appliances may be deployed in various body lumens for a variety of purposes. Stents may be deployed, for example, in the circulatory system for a variety of therapeutic purposes including the treatment of occlusions within the lumens of that system. The current disclosure may be applicable to stents or other medical appliances designed for the central venous (“CV”) system, peripheral vascular (“PV”) stents, abdominal aortic aneurism (“AAA”) stents, bronchial stents, esophageal stents, biliary stents, coronary stents, gastrointestinal stents, neuro stents, thoracic aortic endographs, or any other stent or stent graft.

Further, the present disclosure may be equally applicable to other prosthesis such as grafts. Any medical appliance comprised of materials herein described may be configured for use or implantation within various areas of the body, including vascular, cranial, thoracic, pulmonary, esophageal, abdominal, or ocular application. Examples of medical appliances within the scope of this disclosure include, but are not limited to, stents, vascular grafts, stent grafts, cardiovascular patches, reconstructive tissue patches, medical device coverings and coatings, blood filters, artificial organs, and so forth. For convenience, many of the specific examples included below reference stents. Notwithstanding any of the particular medical appliances referenced in the examples or disclosure below, the disclosure and examples may apply analogously to any prostheses or other medical appliance.

As used herein, the term stent refers to a medical appliance configured for use within a bodily structure, such as within a body lumen. A stent may comprise a scaffolding or support structure, such as a frame, and/or a covering. Thus, as used herein, “stent” refers to both covered and uncovered scaffolding structures.

It will be readily understood that the components of the embodiments as generally described and illustrated in the figures herein could be arranged and designed in a wide variety of configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

The phrases “connected to” and “coupled to” refer to any form of interaction between two or more entities, including mechanical. Two components may be coupled to each other even though they are not in direct contact with each other. For example, two components may be coupled to each other through an intermediate component.

The directional terms “proximal” and “distal” are used herein to refer to opposite locations on a medical device. The proximal end of the device is defined as the end of the device closest to the practitioner when the device is in use by the practitioner. The distal end is the end opposite the proximal end, along the longitudinal direction of the device, or the end furthest from the practitioner.

Again, though the embodiments specifically described below may reference a stent deployment device specifically, the concepts, devices, and assemblies discussed below may be analogously applied to deployment of a wide variety of medical appliances in a wide variety of locations within the body.

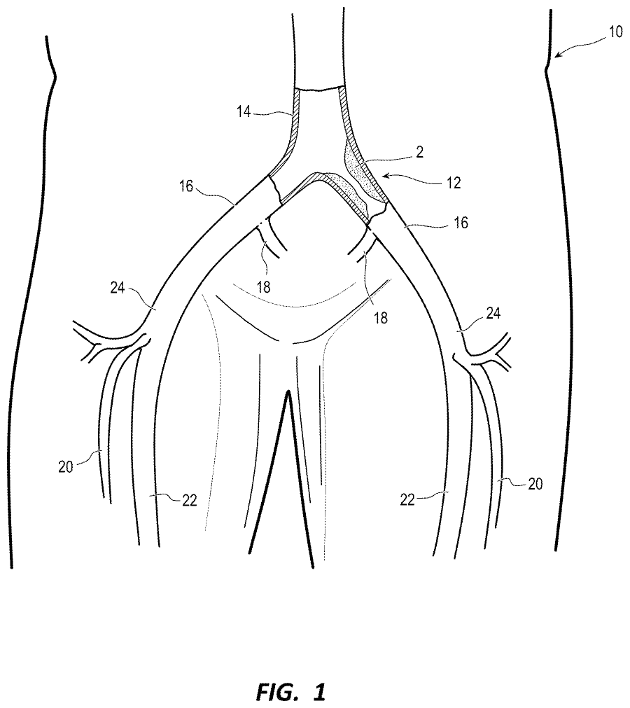

Aortoiliac occlusive disease is a type of peripheral arterial disease which is caused by occlusion of an artery due to atherosclerotic plaque buildup, thrombosis, or embolism. Peripheral arterial disease normally affects the distal femoral artery, but aortoiliac disease is caused by occlusion of the infrarenal aorta and beyond. The aorta branches into the right and left common iliac arteries. Aortoiliac disease can include the common iliac arteries and its branches. Depending on the underlying cause, aortoiliac disease can present acutely or chronically. Acute causes include thrombosis and embolism, while chronic cause include atherosclerotic plaque formation. FIG. 1 is a simplified model aortoiliac occlusive disease of a patient illustrating a stenosis (blockage) 2 in a common iliac artery 12 of a patient 10. For reference, an infrarenal aorta 14, external iliac arteries (right and left) 16, internal iliac arteries (right and left) 18, deep femoral arteries (right and left) 20, superficial femoral arteries (right and left) 22, common femoral arteries (right and left) 24 are illustrated.

Aortoiliac occlusive disease may be classified into various types of lesions. Type A lesions include unilateral or bilateral stenosis of the common iliac artery 12 or a short (less than 3 cm) lesions in the external iliac artery 16. The stenosis 2 illustrated in FIG. 1 is a Type A lesion.

Type B lesions include short (less than 3 cm) stenosis of the infrarenal aorta 14, a unilateral occlusion in the common iliac artery 12, a single or multiple stenosis totaling 3-10 cm involving the external iliac artery 16 but not extending into the common femoral artery 24, and unilateral occlusion in the external iliac artery 16 not involving the origins of the internal iliac artery 18 or the common femoral artery 24.

Type C lesions include bilateral common iliac artery 12 occlusions, bilateral stenosis in the external iliac artery 16 from 3-10 cm long not extending in to the common femoral artery 24, unilateral stenosis in the external iliac artery 16 extending in the common femoral artery 24, unilateral occlusion in the external iliac artery 16 that involves the origins of the internal iliac artery 18 and/or the common femoral artery 24, and heavily calcified unilateral occlusion in the external iliac artery 16 with or without involvement of origins of the internal iliac artery 18 and/r the common femoral artery 24.

Type D lesions include first an occlusion in the infrarenal artery 14, second, diffuse disease involving the infrarenal aorta 14 and both external iliac arteries 16, third, diffuse multiple stenosis involving the unilateral common iliac artery 12, the external iliac arteries 16, common femoral arteries 24, fourth, unilateral occlusions of both common iliac artery 12 and the external iliac arteries 17, fifth, bilateral occlusions of the external iliac arteries 16, and sixth, iliac stenosis in patients with abdominal aortic aneurysm requiring treatment and not amenable to endograft placement or other lesions requiring open aortic or iliac surgery.

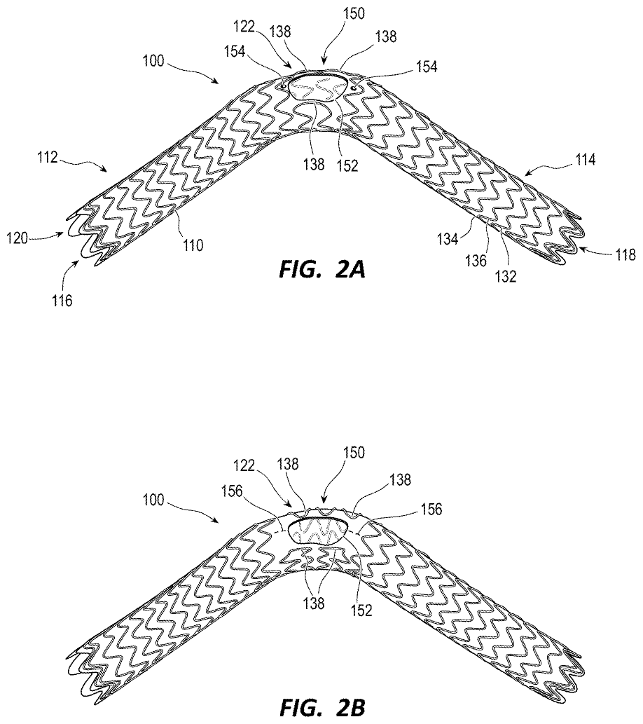

FIG. 2A illustrates a stent 100 in an expanded configuration. The stent 100 may be crimped into a relatively low-profile configuration for delivery. For ease of the disclosure, the stent 100 is described in relation to treating aortoiliac occlusive disease (types A-D), however, the stent 100 is not so limited and may be used in treat vascular diseases in other locations in the patient 10. For example, the stent 100 may be used to for vein to artery anastomosis, treat an anastomosis at a fistula, treat vessels in a patients arm, and the like.

The stent 100 has a body 110 with a tubular structure with a first end portion 112, a second end portion 114, and a central portion 122. The first end portion 112 may have a first opening 116 and the second end portion 114 may have a second opening 118 opposite the first opening 116, and a lumen 120 that extends from the first opening 116 to the second opening 118.

The body 110 of the stent 100 may include a scaffolding structure or frame 130 and a covering 140 disposed over at least a portion of the frame 130. The frame 130 is configured to resist radial compression when the stent 100 is disposed in a lumen of a patient. In some embodiments, the frame 130 may consist of a single continuous wire 132 forming a plurality of helixes that wrap around forming the lumen 120 of the body 110. In some embodiments, the frame 130 may comprise more than one wire 132. The wire 132 may be comprised of Nitinol (ASTM F2063), or other suitable materials. The frame 130 may be shape set (e.g., heat set) to a particular shape such that when the stent 100 is deployed in the vasculature, the stent 100 conforms to the shape set configuration which may mimic the shape of the vasculature where the stent 100 is implanted.

In the illustrated embodiment of FIG. 2A, the wire 132 may be shaped in a wave-type configuration, the waves defining apexes 134 and arms 136 of the frame 130. The length of each arm 136 of the wire 132 may vary in length and may vary in length along a longitudinal length of the frame 130 itself. The apexes 134 are longitudinally separated along the longitudinal length of the body 110. Along some portions of the body 110, the adjacent helixes of the wire 132 in the longitudinally direction are evenly spaced. Along other portions of the body 110, the space between adjacent helixes in the longitudinal direction are not evenly spaced. For example, the space between adjacent helixes near the first opening 116 and the second opening 118 may comprise multiples helixes relatively close to each other to reinforce the frame 130 at the first opening 116 and the second opening 118. In some embodiments, adjacent helixes may touch each other and/or be mechanically coupled to each other. However, the present disclosure is not so limited, and the wire 132 may have a variety of different shapes and sizes to form the frame 130 to support the body 110 of the stent 100.

The frame 130 may be designed such that the central portion 122 is “harder” than the first end portion 112 and the second end portion 114. The “hardness” of the frame 130 refers to the relative strength of the structure (e.g., its compressibility). A harder portion of the frame 130 will have greater strength (i.e., exert a greater radial outward force) than a softer portion. In one embodiment, the central portion 122 is harder than the first end portion 112 and the second end portion 114 which are relatively softer. Further, the frame 130 may be configured to be flexible to facilitate the ability of the stent 100 to conform to the native anatomy at which the stent 100 is configured for use. Similarly, covered devices may be configured with covers 140 which conform to the native anatomy at a therapy site, such as the common iliac artery 12.

Additionally, the frame 130 may be configured to allow the entire stent 100 to be crimped into a relatively low-profile configuration for delivery. For example, devices of a certain diameter or constrained profile are more feasible for delivery at certain vascular or other access points than others. For example, in many instances, a device configured for insertion via the radial artery may be relatively smaller than devices configured for insertion via the generally larger femoral artery. A frame may be configured to be crimped into a particular profile to enable potential access at various or desired access points. Similarly, devices having no frame may be configured to be disposed in a particular profile to facilitate access and delivery. Once a device is positioned within the body it may be expanded or deployed in a number of ways, including use of self-expanding materials and configurations. Additionally, some configurations may be designed for expansion by a secondary device, such as a balloon.

As discussed above, the body 110 comprises a covering 140 that at least partially covers the frame 130. In the illustrated embodiment of FIG. 2, an outer portion of the frame 130 is visible and an inner portion of the frame 130 may be encompassed in the covering 140. In some embodiments, the frame 130 may be completely disposed within the covering 140. In some embodiments, the inner portion of the frame 130 may be disposed out of the covering 140 and an outer portion of the frame 130 is disposed within the covering 140. In some embodiments, the covering 140 may comprise multiple layers. The covering 140 may be a polymer, multiple layers of the same polymer, or layers of distinct polymers used in combination.

The stent 100 further comprises a single fenestration 150 disposed in a sidewall of the body 110 of the stent 100 between the first opening 116 and the second opening 118 in a length direction of the body 110. In other words, in some embodiments, the single fenestration 150 is even, or flush, with the body 110 of the stent 100 and does not extend outward or inward from the body 110. In other words, the fenestration 150 may comprise an opening that is flush with the body 110 of the stent 100, and may not include projections or skirts that extend inwardly or outwardly. The single fenestration 150 may be disposed in the sidewall of the central portion 122 of the body 110 in a length direction of the body 110 of the stent 100. In the illustrated embodiment, the single fenestration 150 is disposed in a center of the body 110 in the length direction of the body 110 of the stent 100. In some embodiments, the stent 100 is symmetric about a center of the single fenestration 150.

The covering 140 defines an outer edge 152 of the single fenestration 150 and the covering 140 not extend into the single fenestration 150. Accordingly, the covering 140 clearly defines the boundaries or outer edge 152 of the single fenestration 150.

The frame 130 of the body 110 does not extend into the single fenestration 150. As illustrated in FIG. 2A, the wire 132 curves back 138 on itself so that the wire 132 does not extend in to the single fenestration 150. Thus the wire 132 reinforces the outer edge 152 of the single fenestration 150 while not extending into the single fenestration 150. The illustrated embodiment illustrates three separate curve backs 138, one curve back 138 on a later edge (e.g., a bottom edge of the single fenestration 150 in the illustrated embodiment of FIG. 2A) of the single fenestration 150 and two curve backs 138 on an opposite lateral edge (e.g., an upper edge of the single fenestration 150 in the illustrated embodiment of FIG. 2A) of the single fenestration 150. As discussed above, the frame 130 may be a single wire 132 such that the wire 132 needs to curve back on itself to form the frame 130 so that the wire 132 does not extend into the single fenestration 150.

In the illustrated embodiment, the stent 100 has a curved configuration. The curve shape may be described as a V-shape or U-shape. The curve of the illustrated stent 100 is not a compound curve. As discussed above, the frame 130 may comprise Nitinol such that shape of the stent 100 may be shape set to a specific shape. The curve of the stent 100 allows for the stent 100 to conform to the native anatomy at which the stent 100 is configured for use. The shape of the stent 100 in the illustrated embodiment of FIG. 2A may be place at a bifurcation of the common iliac artery 12. However, the current disclosure is not so limited, and the stent 100 may have a variety different shapes, such as straight, or more complex curves, such as a compound curve. Additional shapes are discussed below in conjunction with other embodiments.

In some embodiments, the stent 100 may flare along the longitudinal length of the stent 100. For example, a diameter of the central portion 122 is greater than a diameter of the first end portion 112 and the second end portion 114. In some embodiments, that may be reversed and the diameter of the center portion 122 is less than the diameter of the first end portion 112 and the second end portion 114. In some embodiments, the diameters of the first end portion 112 and the second end portion 114 are the same, but in other embodiments, the diameters of the first end portion 112 and the second end portion 114 are different. In the illustrated embodiment of FIG. 2A, the stent 100 has a constant diameter along the entire longitudinal length of the stent 100.

In some embodiments, the body 110 of the stent 100 may comprise one or more marker bands 154. The marker bands 154 may be used to indicate the location of the single fenestration 150 to the user. The marker bands 154 may also be used to help orientate the single fenestration 150 to the proper location in the vasculature. In some embodiments, a marker band 154 may be disposed on the first end portion 112 of the single fenestration 150. In some embodiments, a marker band 154 may be disposed on the second end portion 114 of the single fenestration 150. In some embodiments, marker bands 154 may be disposed on the first end portion 112 and the second end portion 114 of the single fenestration 150. In some embodiments, the marker bands 154 may be disposed on lateral sides of the single fenestration 150. In the illustrated embodiment, the marker bands 154 are distinct points, however, the present disclosure is not so limited. The marker bands 154 may be variety of different shapes and sizes. For example, in some embodiments, the marker band 154 may encircle the entire outer edge 152 or a portion of the outer edge 1552 of the single fenestration 150.

The size of the single fenestration 150 is designed to support the desired flow through the stent 100. In some embodiments, the size of the single fenestration 150 may be dynamic that that the single fenestration 150 may be enlarged based on the desired flow through the stent 100. In other words, the size of the single fenestration 150 is adjustable by the user. For example, as illustrated in FIG. 2B, the body 110 of the stent 100 may comprise a plurality of perforations 156 that extends in a length direction of the stent 100 adjacent to the proximal side of the single fenestration 150 and the distal side of the single fenestration 150. The perforations 156 may be spilt to expand the overall size of the single fenestration 150. In some embodiments, the perforations 156 may be spilt by using an expandable balloon to expand the stent 100 and simultaneously spilt the perforations 156 to enlarge the single fenestration 150. The amount of force to split the perforations 156 may be varied along the length of the perforations 156. For example, perforations 156 closer to the single fenestration 150 may spilt easier than perforations 156 further away from the single fenestration 150. For example, shorter perforations 156 may require more force to open than longer perforations 156. In some embodiments, the perforations 156 may be the same length, whereas in other embodiments, the length of the perforations 156 may vary. In some embodiments, the space between adjacent perforations 156 may be the same, whereas in other embodiments, the space between adjacent perforations 156 may vary, with the larger space between adjacent perforations 156 requiring more force to spilt. The perforations 156 furthest away from the single fenestration 150 may be reinforced so that the single fenestration 150 does not extend past the last perforations 156. Accordingly, the single fenestration 150 is a dynamic opening in which a user may be able to control the actual size of the single fenestration 150 based on the circumstances and the location where the stent is implanted.

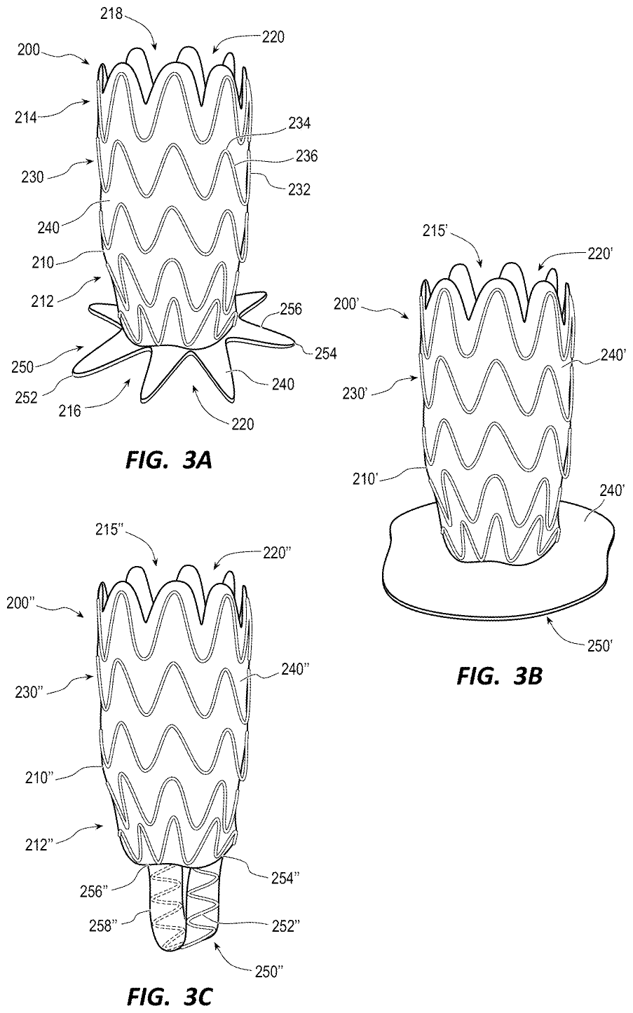

FIG. 3A illustrates a trunk stent 200 that may be used in conjunction with the stent 100. The trunk stent 200 has a body 210 with a tubular structure. The body 210 includes a first end portion 212 and a second end portion 214. The first end portion 212 may have a first opening 216 and the second end portion 214 may have a second opening 218 with a lumen 220 that extends from the first opening 216 to the second opening 218.

The body 210 of the trunk stent 200 may include a scaffolding structure or frame 230 and a covering 240 disposed over at least a portion of the frame 230. In some embodiments, the frame 230 may consist of a single continuous wire 232 forming a plurality of helixes that wrap around the lumen 220 of the body 210. In some embodiments, the frame 230 may comprise more than one wire 232.

In the illustrated embodiment of FIG. 3A, the wire 232 may be shaped in a wave-type configuration, the waves defining apexes 234 and arms 236 of the frame 230. The length of each arm 236 of the wire 232 may vary in length and may vary in length along a length of the frame 230 itself. The helixes are longitudinally separated along the longitudinal length of the body 210. Along some portions of the body 210, the adjacent helixes of the wire 232 in the longitudinally direction are evenly spaced. Along other portions of the body 210, the space between adjacent helixes in the longitudinal direction are not evenly spaced. For example, the space between adjacent helixes near the first opening 216 may comprise multiples helixes relatively close to each other to reinforce the frame 230 at the first opening 216. In some embodiments, the second opening 218 may be similarly reinforced. However, the present disclosure is not so limited, and the wire 232 may have a variety of different shapes and sizes to form the frame 230 to support the body 210 of the stent 200.

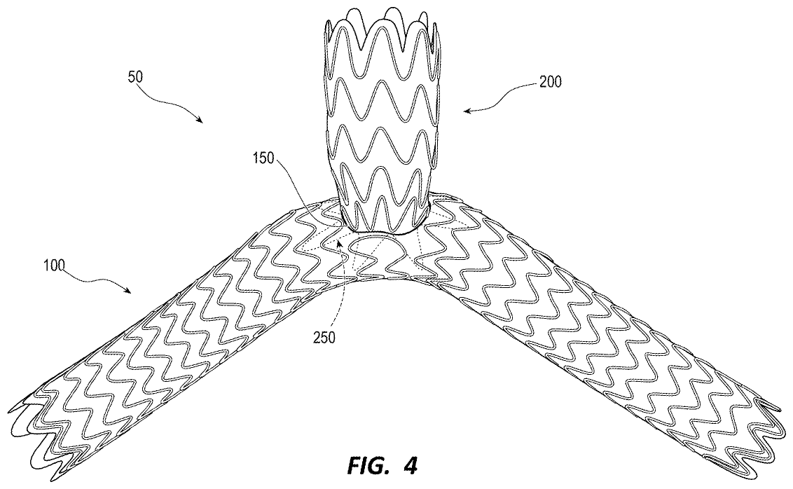

The trunk stent 200 may further comprise an anchor 250. FIG. 3A illustrates one embodiment of the anchor 250 that comprises a wire 252 that may be shaped in a wave-type configuration with a plurality of projections extending radially from the first end portion 212. The waves of the anchor 250 defining apexes 254 and arms 256. The wire 252 of the anchor 250 may be the same wire 232 as the frame 230. The projections of the anchor 250 extend radially outward from the body 210 of the trunk stent 200. In the illustrated embodiment, the projections of the anchor 250 are substantially perpendicular to the body 210 of the trunk stent 200. The waves of the anchor 250 provide an attachment mechanism to secure the trunk stent 200 with the anchor disposed within the lumen 120 of the stent 100 as discussed below in conjunction with FIG. 4.

FIG. 3B depict an embodiment of a trunk stent 200′ that resembles the trunk stent 200 described above in certain respects. Accordingly, like features are designated with like reference numerals, with an apostrophe added to the reference number. For example, the embodiment depicted in FIG. 3B includes a body 210′ that may, in some respects, resemble the body 210 of FIG. 3A. Relevant disclosure set forth above regarding similarly identified features thus may not be repeated hereafter. Moreover, specific features of the trunk stent 200 and related components shown in FIG. 3A may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the trunk stent 200′ and related components depicted in FIG. 3B. Any suitable combination of the features, and variations of the same, described with respect to the trunk stent 200 and related components illustrated in FIG. 3A, can be employed with the trunk stent 200′ and related components of FIG. 3B, and vice versa. This pattern of disclosure applies equally to further embodiments depicted in subsequent figures and described hereafter, wherein the leading digits may be further incremented.

Similar to the trunk stent 200 of FIG. 3A, the trunk stent 200′ of FIG. 3B further comprises an anchor 250′. The anchor 250′ includes a flange that extends radially outward from a body 210′ of the trunk stent 200′. In the illustrated embodiment, the anchor 250′ extends substantially perpendicular to the body 210′ of the trunk stent 200′. The material of the flange of the anchor 250′ may be the same material as a covering 240′ of the body 210′. While not seen, the flange of the anchor 250′ may comprise a wire that reinforces the flange of the anchor 250′. In some embodiments, the wire may be shaped in a wave-type configuration, similar to the wire 232′ of the anchor frame 230′. In some embodiments, the wire may be extend along a perimeter of the anchor 250′. The anchor 250′ provide an attachment mechanism to secure the trunk stent 200′ to the stent 100 as discussed below in conjunction with FIG. 4.

FIG. 3C depicts an embodiment of a trunk stent 200″ that includes a body 210″ with a frame 230″ and a cover 240″, and an anchor 250″ that acts as an attachment mechanism to secure the trunk stent 200″ to the stent 100. The anchor 250″ includes a loop 258″ that extends from the body 210″. A first end 254″ of the loop 258″ may be coupled to the body 210″ a lateral edge near an opening 216″ at a first end portion 212″ and a second end 256″ may couple to the body 210″ near an opposing lateral edge as the first end 254″. The loop 258″ is configured to slide over the stent 100 in an unexpanded or partially expanded configuration. As the stent 100 is expanded, the loop 258″ is secured to the stent 100 via a friction fit. The material of the loop 258″ of the anchor 250″ may be the same material as the covering 240″ of the body 210″. In some embodiments, the loop 258″ may comprise a wire 252″ that reinforces the loop 258″. In some embodiments, the wire 252″ may be shaped in a wave-type configuration.

FIG. 4 illustrates a stent system 50 that includes the stent 100 and the trunk stent 200 coupled together. The trunk stent 200 may be inserted through the single fenestration 150 and the anchor 250 of the trunk stent 200 couples the trunk stent 200 to the stent 100. The anchor 250 is illustrated by phantom lines in FIG. 4.

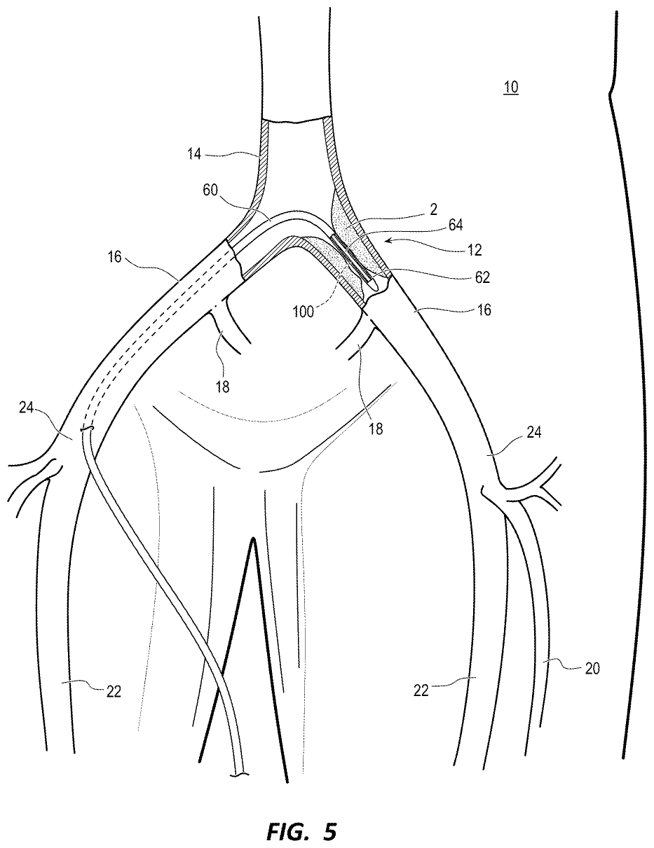

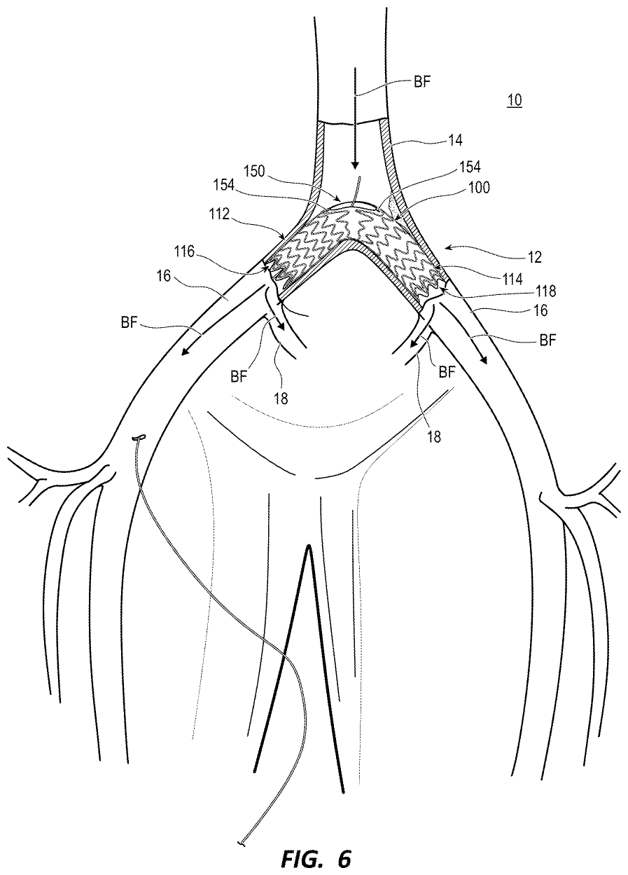

FIGS. 5-8 depicts a process of implanting the stent system 50 at the bifurcation of the common iliac artery 12 of the patient 10. The stent system 50 may be delivered via a delivery device (not shown). The delivery device may include delivery catheter assembly 60 that is advanced to a treatment location within the patient's body from an insertion site. The delivery catheter assembly 60 may be advanced over a guidewire (not shown in FIG. 5, see 66 in FIG. 6). In the illustrated embodiment of FIG. 5, the delivery catheter assembly 60 is advanced from an insertion site 26 in the common femoral artery 24 to the bifurcation of the common iliac artery 12 with a stenosis 2. The stent 100 is advanced to the stenosis 2 in a linear compressed configuration and then deployed. For ease of explanation, a portion of a sheath 62 is removed so show the stent 100 disposed between the sheath 62 and a catheter 64 of the delivery catheter assembly 60. To deploy the stent 100, the sheath 62 is pulled in the proximal direction to expose the stent 100. The stent 100 may be a self-expanding stent and the stent 100 may begin to expand radially outward when the sheath 62 to pulled away. In some embodiments, the stent 100 is expanded with an expandable member, such as a balloon, that expands the stent 100 radially outward.

The stent 100 is positioned such that the single fenestration 150 is positioned at the bifurcation of the common iliac artery 12 at the infrarenal aorta 14 as illustrated in FIG. 6. The delivery catheter assembly 60 may be removed after the stent 100 is deployed, however, the guidewire 66 may remain. The first end portion 112 may be disposed in the right external iliac artery 16, the second end portion 114 may be disposed in the left external iliac artery 16, and the central portion 122 may be disposed at the bifurcation. The marker bands 154 may help the medical practitioner to align the single fenestration 150 with the infrarenal aorta 14 so that blood may flow from the infrarenal aorta 14 into the stent 100. As discussed above, the single fenestration 150 is sized to allow flow from the infrarenal aorta 14 to the stent and to the common iliac artery 12. The single fenestration 150 may be dynamic in that the size of the single fenestration 150 may be enlarged based on size of the patient's infrarenal aorta 14 and common iliac artery 12.

A length of the stent 100 is sized so that the stent 100 does not block branch vessels, such as the internal iliac arteries 18 to allow the internal iliac arteries 18 to remain open to allow blood flow. In other words, the first opening 116 is proximal to the right internal iliac artery 18 and the second opening 118 is proximal to the left internal iliac artery 18.

In this configuration, the single fenestration 150 is an inflow port allowing bodily fluids or blood from the infrarenal aorta 14 to flow into the stent 100 and the first opening 116 and the second opening 118 are outflow ports allowing bodily fluids or blood to flow out of the stent 100. In other words, blood flows into the stent 100 from the single fenestration 150 and blood flows out of the stent 100 through the first opening 116 and the second opening 118 as shown by the arrows labeled BF.

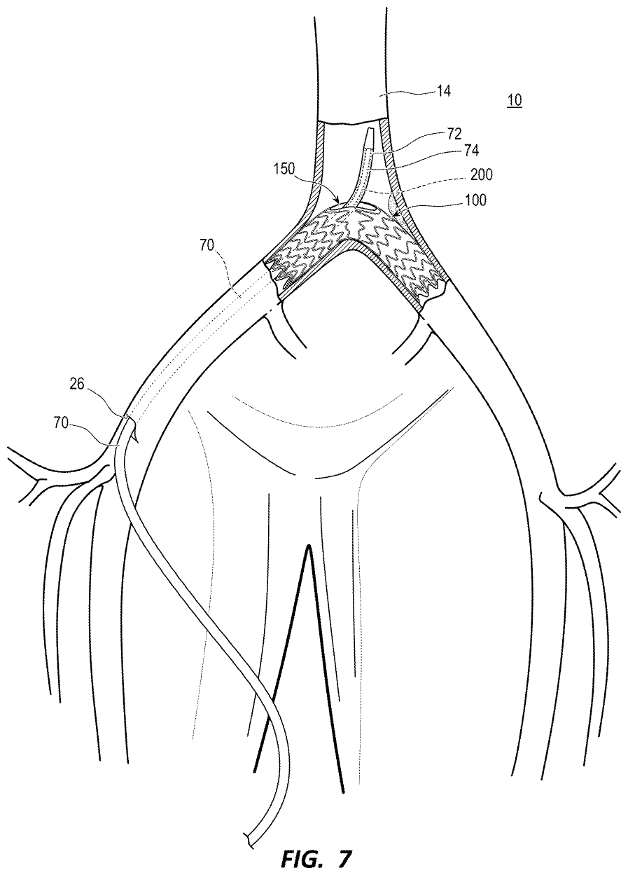

In some circumstances, the stent 100 may be augmented by coupling the trunk stent 200 to the stent 100. FIG. 7 illustrates a trunk stent delivery catheter assembly 70 advanced over the guidewire 66 from the insertion site 26 through the single fenestration 150 at the infrarenal aorta 14. For ease of explanation, a portion of a sheath 72 is removed so show the trunk stent 200 disposed between the sheath 72 and a catheter 74 of the delivery catheter assembly 70. To deploy the trunk stent 200, the sheath 72 is pulled in the proximal direction to expose the trunk stent 200. The trunk stent 200 may be a self-expanding stent and the trunk stent 200 may begin to expand radially outward when the sheath 72 to pulled away. In some embodiments, the trunk stent 200 is expanded with an expandable member, such as a balloon that expands the trunk

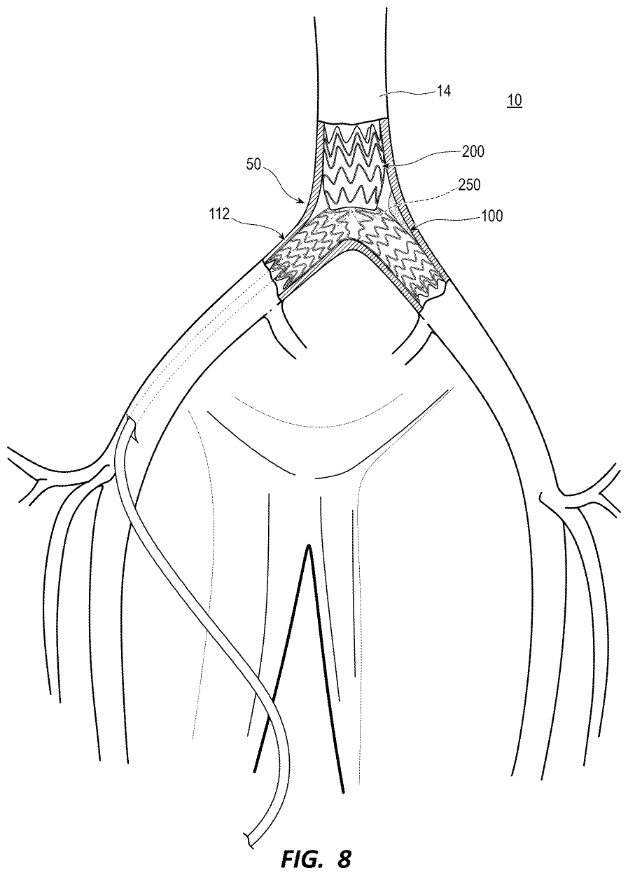

stent 200 radially outward. FIG. 8 illustrates the trunk stent 200 deployed and coupled to the stent 100 via the anchor 250 to form the stent system 50. The trunk stent 200 may be useful for Type B lesions and Type D lesions that include blockages in the infrarenal aorta 14. The delivery catheter assembly 70 may be removed after the stent system 50 is fully deployed.

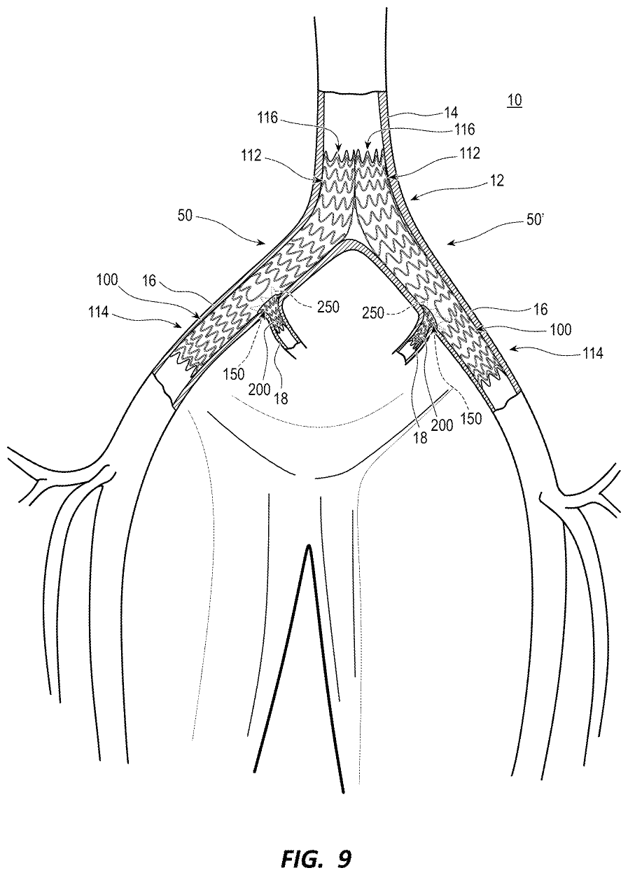

FIG. 9 illustrates another method of using the stent system 50 to treat blockages in the common iliac artery 12 of the patient 10. In the illustrated embodiment, two stent system 50 and 50′ may be used to treat blockages in the common iliac artery 12. Each stent system 50 and 50′ includes the stent 100 and the trunk stent 200. The first end portion 112 of each stent system 50 and 50′ may be disposed in the infrarenal aorta 14 with the first opening 116 configured to receive blood flow from the infrarenal aorta 14. As shown in the illustrated embodiment, both first end portions 112 of each stent system 50 and 50′ is able to be disposed within the infrared aorta 14. The first end portions 112 of each stent system 50 and 50′ may be sized such that each is able to fit within the infrared aorta 14.

The second end portion 114 of each stent system 50 and 50′ is disposed in corresponding portions of the common iliac stent 12. For example, the second end portion 114 of the stent system 50 is disposed in the right external iliac artery 16 and the second end portion 114 of the stent system 50′ is disposed in the left external iliac artery 16.

The single fenestration 150 of each stent system 50 and 50′ may be orientated such that the single fenestration 150 faces the corresponding internal iliac artery 18. For example, the single fenestration 150 of stent system 50 is open to the right internal iliac artery 18 and the single fenestration 150 of stent system 50′ is open to the left internal iliac artery 18. Corresponding trunk stents 200 may be placed within the corresponding single fenestration 150 of each stent system 50 and 50′ such that the trunk stent 200 extends into the corresponding internal iliac artery 18. The trunk stents 200 of each stent system 50 and 50′ are coupled to the corresponding stent 100 by the anchor 250 of each trunk stent 200. In the illustrated embodiment, the single fenestration 150 and the trunk stent 200 may be smaller than in the embodiment shown in FIG. 8 based on the location of the single fenestrations 150 and trunk stent 200. In other words, the single fenestration 150 and the trunk stent 200 of FIG. 9 because they are disposed in the internal iliac arteries 18 rather than the infrarenal aorta artery 14. However, the same sized single fenestration 150 and trunk stent 200 may be used and the trunk stent 200 may not expand as big as the trunk stent illustrated in FIG. 8.

While FIG. 9 illustrates two stent systems 50 and 50′, in some embodiments, a single stent system may be implanted in the illustrated manner. For example, a single stent system 50 may be implanted in the left external iliac artery 16 or in the right external iliac artery 16.

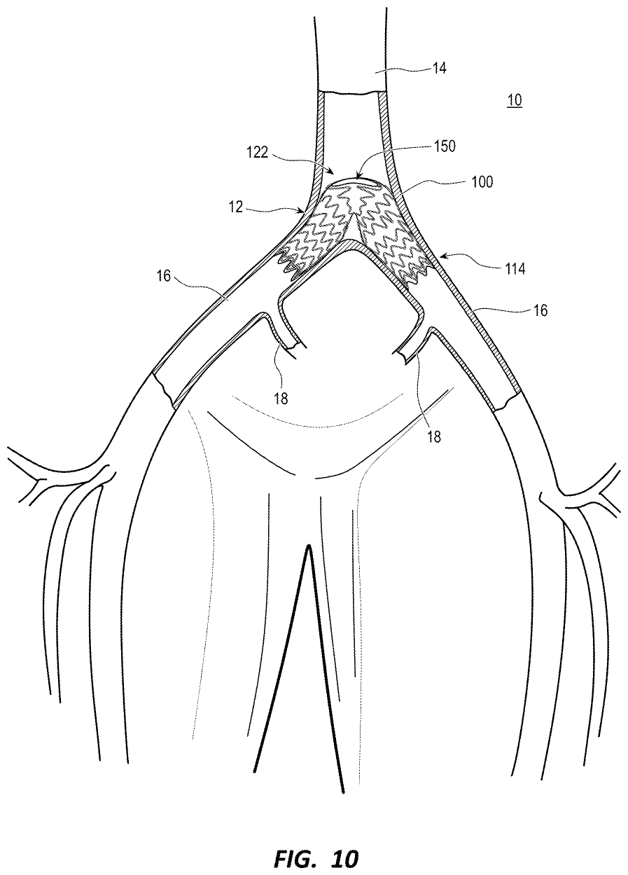

FIG. 10 illustrates the stent 100 implanted in the common iliac artery 12 of the patient 10 in a different implantation position than illustrated in FIG. 6. The stent 100 comprises a constant diameter from the first end portion 112 to the second end portion 114. In this illustrated implantation position, the single fenestration 150 is implanted further into the infrarenal aorta 14 so that the central portion 122 folds up on itself. The first end portion 112 is disposed in the left external iliac artery 16 and the second end portion 114 is disposed in the left external iliac artery 16. The first end portion 112 does not extend past the right internal iliac artery 18 and the second end portion 114 does not extend part the left internal iliac artery 18. In some embodiments, the trunk stent 200 may be used in this particular implantation of the stent 100.

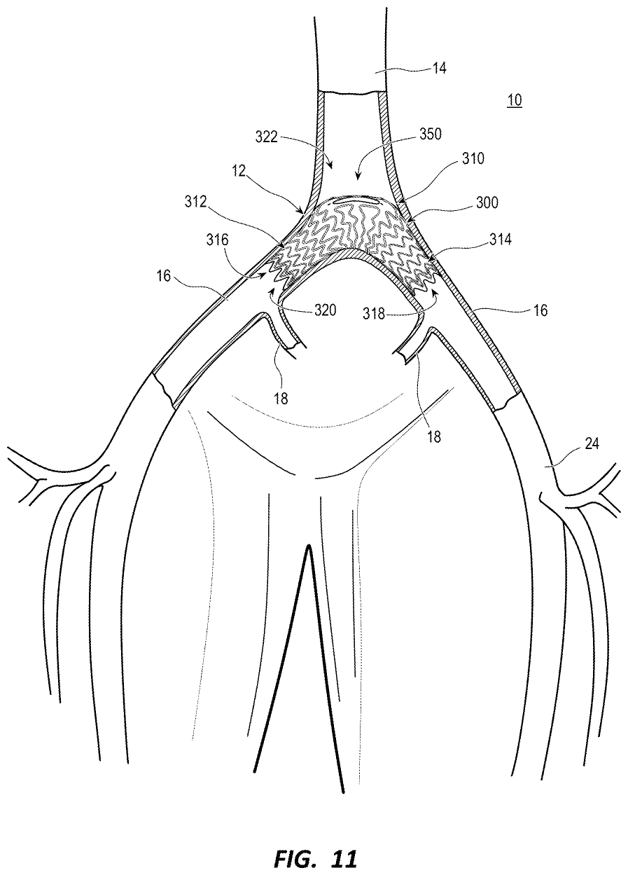

FIG. 11 illustrates an embodiment of a stent 300 that resembles the stent 100 described above in certain respects. Accordingly, like features are designated with like reference numerals, with the leading digits incremented to “3.” For example, the embodiment depicted in FIG. 11 includes a body 310 that may, in some respects, resemble the body 110 of FIGS. 2 and 4. Relevant disclosure set forth above regarding similarly identified features thus may not be repeated hereafter. Moreover, specific features of the stent 100 and related components shown in FIGS. 2 and 4 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the stent 300 and related components depicted in FIG. 11. Any suitable combination of the features, and variations of the same, described with respect to the stent 100 and related components illustrated in FIGS. 2 and 4, can be employed with the stent 300 and related components of FIG. 11, and vice versa. This pattern of disclosure applies equally to further embodiments depicted in subsequent figures and described hereafter, wherein the leading digits may be further incremented.

The stent 300 of FIG. 11 is implanted in the common iliac artery 12 of the patient 10. The stent 300 does not have a constant diameter along the longitudinal length of the stent 300. The stent 300 has a body 310 with a first end portion 312, a second end portion 314, a first opening 316, a second opening 318, a lumen 320 that extends form the first opening 316 to the second opening 318, and a central portion 322. A single fenestration 350 is disposed in a sidewall of the central portion 322. A diameter of the central portion 322 is greater than a diameter of the first end portion 312 and the second end portion 314.

Due to the structure of the stent 300, when the stent 300 is implanted in the common iliac artery 12, the single fenestration may be advanced into the infrarenal aorta 14. The larger diameter of the central portion 322 allows for the stent 300 to extend into the infrarenal aorta 14 from the common iliac artery 12. The first end portion 312 is disposed in the left external iliac artery 16 and the second end portion 314 is disposed in the left external iliac artery 16. The first end portion 312 does not extend past the right internal iliac artery 18 and the second end portion 314 does not extend part the left internal iliac artery 18. In some embodiments, the trunk stent 200 may be used with stent 300.

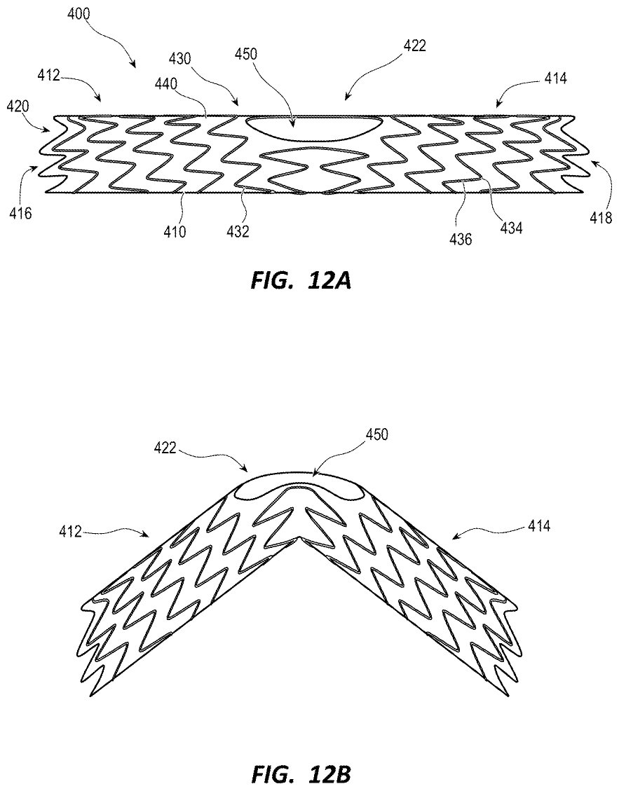

FIGS. 12A and 12B illustrate a stent 400 according to one embodiment. The stent 400 has a body 410 with a first end portion 412, a second end portion 414, a first opening 416, a second opening 418, a lumen 420 that extends form the first opening 416 to the second opening 418, a central portion 422, and a single fenestration 450.

The body 410 of the stent 400 may include a scaffolding structure or frame 430 and a covering 440 disposed over at least a portion of the frame 430. In some embodiments, the frame 430 may consist of a single continuous wire 432 forming a plurality of helixes that wrap around the lumen 420 of the body 410. In some embodiments, the frame 430 may comprise more than one wire 132.

FIG. 12A illustrates the stent 400 is a linear configuration and FIG. 12B illustrates the stent 400 in a curved configuration, such as a V-shape or U-shape. The frame 430 may be shape set (e.g., heat set) to a particular shape such that when the stent 400 is deployed in the vasculature, the stent 400 conforms to the shape set configuration. For example, the stent 400 may be shape set to the shape illustrated in FIG. 12B so that the stent 400 conforms to the shape of the common iliac artery 12 at the bifurcation of the common iliac artery 12. FIG. 12A illustrates the stent 400 is the linear configuration before it is compressed so that the stent 400 may be delivered via the delivery catheter assembly 60. The stent 400 may be compressed in the linear configuration so that it may be delivered through a lumen of the delivery catheter assembly 60.

In the illustrated embodiment of FIGS. 12A and 12B, the wire 432 may be shaped in a wave-type configuration, the waves defining apexes 434 and arms 436 of the frame 430. The length of each arm 436 of the wire 432 may vary in length and may vary in length along a length of the frame 430 itself. The apexes 434 are longitudinally separated along the longitudinal length of the body 410. Along some portions of the body 410, the adjacent helixes of the wire 432 in the longitudinally direction are evenly spaced. Along other portions of the body 410, the space between adjacent helixes in the longitudinal direction are not evenly spaced. For example, the space between adjacent helixes near the first opening 416 and the second opening 418 may comprise multiples helixes relatively close to each other to reinforce the frame 430 at the first opening 416 and the second opening 418. However, the present disclosure is not so limited, and the wire 432 may have a variety of different shapes and sizes to form the frame 430 to support the body 410 of the stent 400.

The orientation of the wire 432 may appear differently based on the configuration of the stent 400. For example, FIG. 12A illustrates the stent 400 in a linear configuration. In the linear configuration, the wire 432 in the first end portion 412 is angled or slanted toward to the central portion 422 and downward and the wire 432 in the second end portion 414 is angled or slanted toward the central portion 422 and downward. Accordingly, when the stent 400 is transitioned to the curved configuration, the wire 432 in first end portion 412 appears to be orthogonal relative to the first end portion 412 and the wire 432 in the second end portion 414 appears to be orthogonal relative to the second end portion 414.

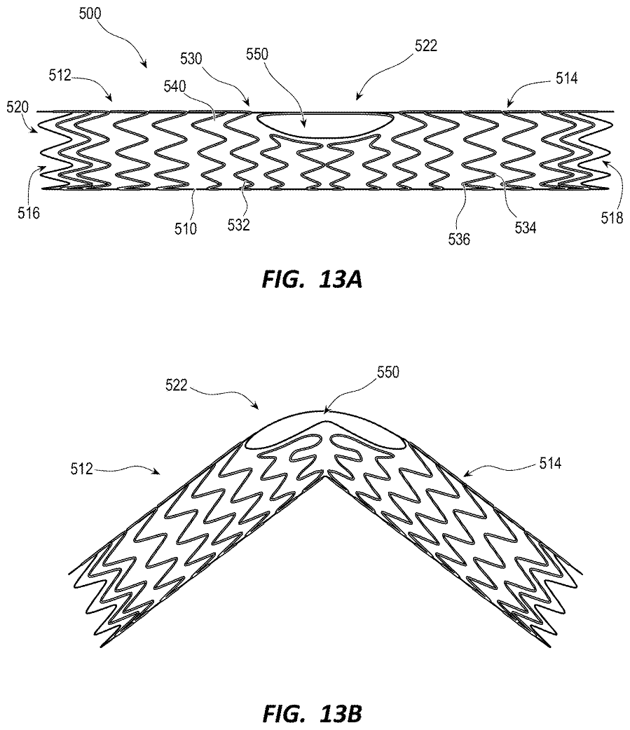

FIGS. 13A and 13B illustrate a stent 500. The stent 500 has a body 510 with a first end portion 512, a second end portion 514, a first opening 516, a second opening 518, a lumen 520 that extends form the first opening 516 to the second opening 518, and a central portion 522.

The body 410 of the stent 400 may include a scaffolding structure or frame 430 and a covering 440 disposed over at least a portion of the frame 430. In some embodiments, the frame 430 may consist of a single continuous wire 432 forming a plurality of helixes that wrap around the lumen 420 of the body 410. In some embodiments, the frame 430 may comprise more than one wire 132.

FIG. 13A illustrates the stent 500 is a linear configuration and FIG. 13B illustrates the stent 500 in a curved configuration, such as a V-shape or U-shape. The frame 530 may be shape set (e.g., heat set) to a particular shape such that when the stent 500 is deployed in the vasculature, the stent 500 conforms to the shape set configuration. For example, the stent 500 may be shape set to the shape illustrated in FIG. 13B so that the stent 500 conforms to the shape of the common iliac artery 12 at the bifurcation of the common iliac artery 12. FIG. 13A illustrates the stent 500 is the linear configuration before it is compressed so that the stent 500 may be delivered via the delivery catheter assembly 60. The stent 500 may be compressed in the linear configuration so that it may be delivered through a lumen of the delivery catheter assembly 60.

In the illustrated embodiment of FIGS. 13A and 13B, the wire 532 may be shaped in a wave-type configuration, the waves defining apexes 534 and arms 536 of the frame 530. The length of each arm 536 of the wire 532 may vary in length and may vary in length along a length of the frame 530 itself. The apexes 534 are longitudinally separated along the longitudinal length of the body 510. Along some portions of the body 510, the adjacent helixes of the wire 532 in the longitudinally direction are evenly spaced. Along other portions of the body 510, the space between adjacent helixes in the longitudinal direction are not evenly spaced. For example, the space between adjacent helixes near the first opening 516 and the second opening 518 may comprise multiples helixes relatively close to each other to reinforce the frame 530 at the first opening 516 and the second opening 518. However, the present disclosure is not so limited, and the wire 532 may have a variety of different shapes and sizes to form the frame 530 to support the body 510 of the stent 500.

In the illustrated embodiment, the length of the arms 536 of some of the helices are different. For example, a few of the helices immediately adjacent to the single fenestration 550 on the first end portion 512 and on the second end portion 514, the arms 536 get smaller as the helix extends laterally away from the single fenestration 550 and the arms get larger as the helix extends laterally toward the single fenestration 550.

The orientation of the wire 532 may appear differently based on the configuration of the stent 500. For example, FIG. 13A illustrates the stent 400 in a linear configuration. In the linear configuration, the length of the arms 536 of some of the helices are different. For example, a few of the helices immediately adjacent to the single fenestration 550 on the first end portion 512 and on the second end portion 514, the arms 536 get smaller as the helix extends laterally away from the single fenestration 550 and the arms get larger as the helix extends laterally toward the single fenestration 550. Accordingly, when the stent 500 is transitioned to the curved configuration, the wire 532 in first end portion 512 appears to be orthogonal relative to the first end portion 512 and the wire 532 in the second end portion 514 appears to be orthogonal relative to the second end portion 514.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure

What is claimed is:

1. A stent comprising:
a body comprising a first end portion with a first opening and a second end portion with a second opening opposite the first opening, and a lumen extending from the first opening to the second opening; and
a single fenestration disposed in a sidewall of the body between the first opening and the second opening,
wherein the single fenestration is disposed in a center portion of the body of the stent in a length direction.

2. The stent of claim 1, wherein the single fenestration is disposed in a center of the body of the stent in the length direction.

3. The stent of claim 1, wherein the stent is system is symmetric about a center of the single fenestration.

4. The stent of claim 1, wherein a diameter of a central portion of the body is greater than a diameter of the body at the first end portion and the diameter of the central portion is greater than a diameter of the body at the second end portion.

5. The stent of claim 1, wherein a diameter of a central portion of the body is less than a diameter of the body at the first end portion and the diameter of the central portion is less than a diameter of the body at the second end portion.

6. The stent of claim 1, wherein an area of the single fenestration is greater than an area of first opening and the area of the single fenestration is greater than an area of the second opening.

7. The stent of claim 1, wherein a diameter of the single fenestration is greater than a diameter of the first opening and the diameter of the single fenestration is greater than a diameter of the second opening.

8. The stent of claim 1, wherein the single fenestration is an inflow port that allows bodily fluid to flow into the stent from the single fenestration and the first opening and the second opening are outflow ports that allow bodily fluid to flow out of the stent.

9. The stent of claim 1, wherein the body comprises a frame configured to resist radial compression when disposed in a lumen of a patient, and
wherein the frame does not extend into the single fenestration.

10. The stent of claim 9, wherein the body comprises a covering disposed on at least a portion of the frame, the covering does not extend into the single fenestration.

11. The stent of claim 1, wherein a size of the single fenestration is adjustable during an expansion of the stent.

12. The stent of claim 1, further comprising a marker band on a proximal or distal side of the single fenestration.

13. The stent of claim 1, further comprising a trunk stent that is configured to extend through the single fenestration and couple to the body.

14. The stent of claim 13, wherein the trunk stent comprises an anchor to secure the trunk stent to the stent.

15. The stent of claim 1, wherein in an expanded configuration, the stent has a curved configuration.

16. A stent comprising:
a body comprising a first end portion with a first opening and a second end portion with a second opening opposite the first opening, and a lumen extending from the first opening to the second opening; and
a single fenestration disposed in a sidewall of the body between the first opening and the second opening,
wherein an area of the single fenestration is greater than an area of first opening and the area of the single fenestration is greater than an area of the second opening.

17. A method of implanting a stent comprising:
advancing a stent to a bifurcation of a common iliac artery near an infrarenal aorta artery;
deploying the stent with a first end portion of the stent deployed in a left external iliac artery and a second end portion of the stent deployed in a right external iliac artery;
adjusting the stent such that a single fenestration disposed in a sidewall of the stent is in fluid communication with a blood flow from the infrarenal aorta artery.

18. The method of claim 17, wherein the first end portion of the stent does not extend to the left internal iliac artery and the second end portion of the stent does not extend to the right internal iliac artery.

19. The method of claim 17, further comprising:
advancing a trunk stent to the stent in the common iliac artery; and
deploying the trunk stent in the single aperture of the stent.

20. The method of claim 17, further comprising coupling the trunk stent to the stent.
